(19) United States
(12) Patent Application Publication
(10) Pub. No.: US 2025/0073161 A1
(43) Pub. Date: Mar. 6, 2025

(54) TOPICAL COMPOSITION AND MANUFACTURING METHOD AND USE THEREOF

(71) Applicant: PANION & BF BIOTECH INC., TAIPEI (TW)

(72) Inventors: Ming-Thau Sheu, Taipei (TW); Yu-De Su, Taipei (TW); Yu-Siang Lin, Taipei (TW)

(73) Assignee: PANION & BF BIOTECH INC., TAIPEI (TW)

(21) Appl. No.: 18/826,114

(22) Filed: Sep. 5, 2024

Related U.S. Application Data
(60) Provisional application No. 63/536,922, filed on Sep. 6, 2023.

(51) Int. Cl. A61K 9/00; A61K 9/06; A61K 9/12; A61K 31/506; A61K 47/10; A61K 47/12; A61K 47/18; A61K 47/38

(52) U.S. Cl. CPC A61K 9/0014; A61K 9/06; A61K 9/122; A61K 31/506; A61K 47/10; A61K 47/12; A61K 47/186; A61K 47/38

(57) ABSTRACT

A topical composition includes minoxidil, a cosolvent, an α-hydroxy acid and water. The cosolvent includes alcohol. A weight ratio of the alcohol to the water is 1:1.4 to 1:10. A method for manufacturing the topical composition includes the following steps: dissolving the α-hydroxy acid in the water; adding the cosolvent to obtain a mixed solution; and adding the minoxidil to the mixed solution. A use of the topical composition is for treating hair loss or promoting hair growth.

### CROSS-REFERENCE TO RELATED APPLICATION

This application claims the priority benefit of U.S. provisional application Ser. No. 63/536,922, filed on Sep. 6, 2023. The entirety of the above-mentioned patent application is hereby incorporated by reference herein and made a part of this specification.

### BACKGROUND

### Technical Field

The invention relates to a topical composition and a manufacturing method and a use thereof, and in particular relates to a topical composition and a manufacturing method thereof that may reduce skin irritation and prevent skin allergies.

### Description of Related Art

Minoxidil is poorly soluble in water. Currently, the products containing minoxidil on the market basically contain propylene glycol. Propylene glycol facilitates the sufficient solubility of minoxidil in water, thereby enabling products containing minoxidil to incorporate approximately 2% or 5% of minoxidil. However, according to statistics, about 20% of users who use products containing minoxidil are allergic to propylene glycol, which leads to increased dandruff, increased skin itching, increased greasiness, and in severe cases, increased hair loss. Furthermore, the statistics indicate that the proportion of propylene glycol allergy is higher among Asian populations compared to Western populations.

### SUMMARY

A topical composition and a manufacturing method thereof, which may reduce skin irritation and prevent skin allergies, are provided in the invention.

The topical composition of the invention includes minoxidil, a cosolvent, an α-hydroxy acid, and water. The cosolvent includes alcohol. A weight ratio of the alcohol to the water is 1:1.4 to 1:10.

In an embodiment of the invention, content of the minoxidil is 1 weight percent concentration to 5 weight percent concentration based on total weight of the topical composition.

In an embodiment of the invention, content of the cosolvent is 18 weight percent concentration to 50 weight percent concentration based on total weight of the topical composition.

In an embodiment of the invention, the cosolvent further includes glycerol. A weight ratio of the alcohol to the glycerol is 1:0.45 to 1:2.

In an embodiment of the invention, the alcohol includes ethanol, isopropyl alcohol, or a combination thereof.

In an embodiment of the invention, content of the α-hydroxy acid is 0.6 weight percent concentration to 5 weight percent concentration based on total weight of the topical composition.

In an embodiment of the invention, a weight ratio of the α-hydroxy acid to the water is 1:10 to 1:100.

In an embodiment of the invention, the α-hydroxy acid includes glycolic acid, lactic acid, acetic acid, citric acid, malic acid, mandelic acid, tartaric acid, or a combination thereof.

In an embodiment of the invention, the topical composition does not include propylene glycol.

In an embodiment of the invention, content of the minoxidil is 1 weight percent concentration to 5 weight percent concentration, content of the cosolvent is 18 weight percent concentration to 50 weight percent concentration, and content of the α-hydroxy acid is 0.6 weight percent concentration to 5 weight percent concentration.

In an embodiment of the invention, the topical composition further includes a thickening agent. Content of the thickening agent is 0.1 weight percent concentration to 3.0 weight percent concentration based on total weight of the topical composition.

In an embodiment of the invention, the thickening agent includes alginate, acacia, carrageenan, guar gum, karaya gum, locust bean gum, tragacanth, xanthan gum, carboxymethylcellulose or salts thereof, hydroxyethyl cellulose, methylcellulose, hydroxypropyl cellulose, hydroxypropyl methylcellulose, hydroxypropyl methylcellulose stearoxy ether, hyaluronic acid or salts thereof, or a combination thereof.

In an embodiment of the invention, the topical composition further includes a foaming agent. Content of the foaming agent is 0.1 weight percent concentration to 3.0 weight percent concentration based on total weight of the topical composition.

In an embodiment of the invention, the foaming agent includes sodium laureth sulfate (or sodium lauryl ether sulfate), sodium dodecyl sulfate, ammonium lauryl sulfate, cocamide foaming agents, PEG-20 oleyl ether, lauryl glucoside, potassium cocoyl glycinate, or a combination thereof.

In an embodiment of the invention, the cocamide foaming agent includes nonionic cocamide DEA, nonionic cocamidopropylamine oxide, zwitterionic cocamidopropyl betaine, zwitterionic cocamidopropyl hydroxysultaine, or a combination thereof.

In an embodiment of the invention, a pH value of the topical composition is 4 to 5.

A manufacturing method of the topical composition includes the following steps: dissolving an α-hydroxy acid in water; adding cosolvent to obtain a mixed solution; and adding minoxidil to the mixed solution. The cosolvent includes alcohol. A weight ratio of the alcohol to the water is 1:1.4 to 1:10.

In an embodiment of the invention, the manufacturing method further includes: adding a thickening agent. Content of the thickening agent is 0.1 weight percent concentration to 3.0 weight percent concentration based on total weight of the topical composition.

In an embodiment of the invention, the manufacturing method further includes: adding a foaming agent. Content of the foaming agent is 0.1 weight percent concentration to 3.0 weight percent concentration based on total weight of the topical composition.

A use of the topical composition is treating hair loss or promoting hair growth.

Based on the above, in the topical composition and the manufacturing method thereof according to one embodiment of the invention, through the use of α-hydroxy acid and alcohol-containing cosolvent, in addition to increasing the solubility of minoxidil in water, skin irritation caused by the topical composition may also be reduced, and skin allergies may also be prevented.

In order to make the above-mentioned features and advantages of the invention comprehensible, embodiments accompanied with drawings are described in detail below.

### DETAILED DESCRIPTION OF DISCLOSED EMBODIMENTS

Hereinafter, embodiments of the invention are described in detail. However, these embodiments are illustrative, and the invention is not limited thereto.

The topical composition in the embodiments of the invention may include minoxidil, a cosolvent, an α-hydroxy acid, and water, but not limited thereto. The topical composition of the embodiment of the invention does not include propylene glycol to reduce skin irritation and prevent skin allergies.

In some embodiments, the pH value of the topical composition may be, for example, 4 to 5, so that the topical composition may be suitable for topical use, but not limited thereto. In some embodiments, the use of the topical composition may be, for example, for treating hair loss (alopecia) or promoting hair growth, but is not limited thereto. In some embodiments, the topical composition may be administered in the form of a solution, a gel, a foam, etc., but not limited thereto.

### Minoxidil

In the embodiments of the invention, minoxidil is the active ingredient of the topical composition. The molecular name of minoxidil is 3-hidroxy-2-imino-6-piperidine-1-pyrimidine-4-amine, and the molecular formula of minoxidil may be as follows:

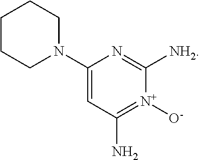 [SMILES: Nc1cc(N2CCCCC2)nc(N)[n+]1[O-]]

Minoxidil is a peripheric vasodilator agent. Specifically, minoxidil may be used to increase the blood flow supply of the scalp, thereby providing more oxygen, blood and nutrients to the hair follicles. Consequently, it may prolong the anagen phase of hair growth through the proliferative and anti-apoptotic effects on dermal papilla cells of the follicles.

In some embodiments, based on the total weight of the topical composition (i.e., when the topical composition is considered as 100 weight percent concentration), the content of minoxidil may be, for example, 1 weight percent concentration (w/w %) to 5 weight percent concentration, preferably 2 weight percent concentration to 5 weight percent concentration, and more preferably 5 weight percent concentration, but not limited thereto.

### Cosolvent

In an embodiment of the invention, the cosolvent may be used to increase the solubility of the minoxidil in water. In an embodiment of the invention, the cosolvent may include alcohol. In some embodiments, the alcohol may be, for example, ethanol, isopropyl alcohol, or a combination thereof, but not limited thereto.

In some embodiments, the weight ratio of alcohol to water is 1:1.4 to 1:10, such as 1:1.4, 1:2, 1:3, 1:4, 1:5, 1:6, 1:7, 1:8, 1:9, 1:10, or any value between any two of these values, but not limited thereto. When the weight ratio of alcohol to water is less than 1:1.4 (e.g., 1:1), the high alcohol content may potentially reduce the permeability of the topical composition and increase its volatility. Furthermore, in practical application, the alcohol may cause skin irritation or allergic reactions. When the weight ratio of alcohol to water is greater than 1:10 (e.g., 1:12), the low alcohol content may potentially reduce the solubility of minoxidil.

In some embodiments, based on the total weight of the topical composition, the content of the cosolvent may be, for example, 18 weight percent concentration to 50 weight percent concentration, such as 18 w/w %, 20 w/w %, 25 w/w %, 30 w/w %, 35 w/w %, 40 w/w %, 45 w/w %, 50 w/w %, or any value between any two of these values, but not limited thereto. When the content of the cosolvent is less than 18 weight percent concentration, the solubility of minoxidil may be reduced or the stability of the topical composition may be reduced. When the content of the cosolvent is greater than 50 weight percent concentration, it may cause skin irritation or toxicity.

In some embodiments, the cosolvent may further include glycerol. The weight ratio of alcohol to glycerol may be, for example, 1:0.45 to 1:2, such as 1:0.45, 1:0.5, 1:1, 1:1.5, 1:2, or any value between any two of these values, but not limited thereto. When the weight ratio of alcohol to glycerol is less than 1:0.45 (e.g., 1:0.1), the glycerol content may be insufficient to enhance the solubility of minoxidil. When the weight ratio of alcohol to glycerol is greater than 1:2 (e.g., 1:3), the high glycerol content may potentially reduce the permeability of the topical composition.

α-hydroxy acid

In an embodiment of the invention, the α-hydroxy acid may be used to increase the solubility of the minoxidil in water. In some embodiments, the α-hydroxy acid may be, for example, glycolic acid, lactic acid, acetic acid, citric acid, malic acid, mandelic acid, tartaric acid, or a combination thereof, but not limited thereto.

In some embodiments, the weight ratio of α-hydroxy acid to water may be, for example, 1:10 to 1:100, such as 1:10, 1:15, 1:20, 1:25, 1:30, 1:35, 1:40, 1:45, 1:50, 1:55, 1:60, 1:65, 1:70, 1:75, 1:80, 1:85, 1:90, 1:95, 1:100, or any value between any two of these values, but not limited thereto. When the weight ratio of α-hydroxy acid to water is less than 1:10 (e.g., 1:9), the α-hydroxy acid content may be too high, potentially resulting in skin irritation or allergic reactions. When the weight ratio of α-hydroxy acid to water is greater than 1:100 (e.g., 1:110), the low α-hydroxy acid content may potentially reduce the solubility of minoxidil.

In some embodiments, based on the total weight of the topical composition, the content of the α-hydroxy acid is 0.6 weight percent concentration to 5 weight percent concentration, such as 0.6 w/w %, 1 w/w %, 2 w/w %, 3 w/w %, 4 w/w %, 5 w/w %, or any value between any two of these values, but not limited thereto. When the content of the α-hydroxy acid is less than 0.6 weight percent concentration, the solubility of minoxidil may be reduced or the stability of the topical composition may be reduced. When the content of the α-hydroxy acid is greater than 5 weight percent concentration, it may cause skin irritation or toxicity.

### Thickening Agent

In some embodiments, the topical composition may further include a thickening agent to increase the viscosity of the topical composition and turn the topical composition into a gel. The thickening agent may be, for example, alginate, acacia, carrageenan, guar gum, karaya gum, locust bean gum, tragacanth, xanthan gum, carboxymethylcellulose or salts thereof, hydroxyethyl cellulose, methylcellulose, hydroxypropyl cellulose, hydroxypropyl methylcellulose, hydroxypropyl methylcellulose stearoxy ether, hyaluronic acid or salts thereof, or a combination thereof, but not limited thereto.

In some embodiments, based on the total weight of the topical composition, the content of the thickening agent is 0.1 weight percent concentration to 3.0 weight percent concentration, such as 0.1 w/w %, 0.2 w/w %, 0.5 w/w %, 1.0 w/w %, 1.5 w/w %, 2.0 w/w %, 2.5 w/w %, 3.0 w/w %, or any value between any two of these values, but not limited thereto.

### Foaming Agent

In some embodiments, the topical composition may further include a foaming agent to turn the topical composition into a foam. The foaming agent may be, for example, sodium laureth sulfate (or sodium lauryl ether sulfate), sodium dodecyl sulfate, ammonium lauryl sulfate, cocamide foaming agent, PEG-20 oleyl ether, lauryl glucoside, potassium cocoyl glycinate, or a combination thereof, but not limited thereto. The cocamide foaming agent may be, for example, nonionic cocamide DEA, nonionic cocamidopropylamine oxide, zwitterionic cocamidopropyl betaine, zwitterionic cocamidopropyl hydroxysultaine, or a combination thereof, but not limited thereto.

In some embodiments, based on the total weight of the topical composition, the content of the foaming agent is 0.1 weight percent concentration to 3.0 weight percent concentration, such as 0.1 w/w %, 0.2 w/w %, 0.5 w/w %, 1.0 w/w %, 1.5 w/w %, 2.0 w/w %, 2.5 w/w %, 3 w/w %, or any value between any two of these values, but not limited thereto.

### Aromatic Agent and Excipient

In some embodiments, the topical composition may further include an aromatic agent and an excipient as inactive ingredients. Potential unpleasant odors may be prevented by adding the aromatic agent to the product. The excipient may include a surfactant/wetting agent, a solubilizer, a penetration enhancer, a preservative, a humectant, a moisturizer, an anti-oxidant, a de-tackifying agent, a conditioning agent, protein, fragrance, and one or more mixtures thereof, but not limited thereto.

The invention also provides a manufacturing method of a topical composition. In an embodiment, the manufacturing method of the topical composition may include, but is not limited to, the following steps and their sequence: firstly, α-hydroxy acid was dissolved in water to obtain an acid solution; next, one or more cosolvents were added to the acid solution to obtain a mixed solution; next, minoxidil was added to the mixed solution and stirred until the minoxidil was dissolved; next, water was added until reaching the final required weight to obtain a topical composition.

In some embodiments, the manufacturing method of the topical composition may further include: a thickening agent was added before adding water until reaching the final required weight and stirred until the thickening agent was dissolved to obtain the topical composition in gel form.

In some embodiments, the manufacturing method of the topical composition may further include: a foaming agent was added before adding water until reaching the final required weight to obtain the topical composition in foam form.

In some embodiments, the manufacturing method of the topical composition may further include: before adding water until reaching the final required weight, a thickening agent was added and stirred until the thickening agent was dissolved; then, a foaming agent was added to obtain the topical composition in foam form.

The topical composition of the invention and the manufacturing method thereof will be described in more detail below with reference to several experiments. Although the following experiments are described, the materials used, their amounts and ratios, processing details, processing procedures, etc. may be appropriately changed without exceeding the scope of the invention. Therefore, the invention should not be interpreted restrictively based on the experiments described below. Hereinafter, embodiments will be enumerated to describe the invention in detail. However, it should be understood that the invention is not limited to the content disclosed in these embodiments.

### <Manufacture of Topical Composition in Solution Form>

### Embodiment 1

First, glycolic acid was dissolved in water to obtain an acid solution. Next, ethanol and glycerol were added to the acid solution to obtain a mixed solution. Then, minoxidil was added to the mixed solution and stirred until the minoxidil was dissolved. Next, water was added until reaching the final required weight to obtain the topical composition of Embodiment 1. Based on the total weight of the topical composition of Embodiment 1 (i.e., when the topical composition of Embodiment 1 is considered as 100 weight percent concentration), the contents of minoxidil, ethanol, glycerol, glycolic acid and water are 5%, 7.5%, 15%, 3% and 69.5% respectively, as shown in Table 1.

### Embodiment 2 to Embodiment 12

The topical compositions of Embodiment 2 to Embodiment 12 are prepared by the same steps as Embodiment 1, with the difference being the types and amounts of ingredients used in each embodiment. The types and amounts of ingredients used in each embodiment are listed in Table 1 or Table 2.

### Comparative Example 1 to Comparative Example 6

The topical compositions of Comparative Example 1 to Comparative Example 6 are prepared by the same steps as Embodiment 1, with the difference being the types and amounts of ingredients used in each comparative example. The types and amounts of ingredients used in each comparative example are listed in Table 2.

### <Testing of Topical Composition in Solution Form>

The topical composition prepared in the above experiment was tested to analyze the solution color, solubility, and stability of the topical composition.

Solution color analysis of topical compositions: when the topical composition is observed to be colorless to light yellow, the topical composition is qualified.

Solubility analysis of the topical composition: after leaving the topical composition at room temperature to stand for 7 days, when it is observed that the topical composition is a clear solution, o is denoted in the table to indicate that the topical composition has good solubility; when it is observed that the topical composition is a turbid solution or solid crystals are present, x is denoted in the table to indicate that the topical composition failed to completely dissolve minoxidil or that minoxidil precipitates within 7 days after the composition is prepared.

Stability test of the topical composition: after leaving the topical composition to stand for 6 months in an environment of 40° C. and 75% relative humidity (RH), high-performance liquid chromatography (HPLC) was used to perform analysis of minoxidil content (Assay) and impurity. When the content of minoxidil (specification: the content of minoxidil reaches 95.0% to 105.0% w/w of the added amount) and impurities (specification: any individual impurity content does not exceed 0.2% (w/w); total impurity content does not exceed 2.0% (w/w)) do not exceed the specification limits, o is used in the table to indicate that the topical composition has good stability; when either the minoxidil content or impurities exceeds the specification limits, x is used in the table to indicate that the topical composition has poor stability.

TABLE 1

| Composition | | Embodiment | | | | | | | | |
|---|---|---|---|---|---|---|---|---|---|---|
| (w/w %) | | 1 | 2 | 3 | 4 | 5 | 6 | 7 | 8 | 9 |
| Minoxidil | | 5 | 1 | 5 | 5 | 5 | 5 | 5 | 5 | 5 |
| Cosolvent | Ethanol | 7.5 | 32 | 12 | 32 | 30 | 10 | 20 | 20 | 20 |
| | Isopropyl | — | — | — | — | — | — | — | — | — |
| | alcohol | | | | | | | | | |
| | Glycerol | 15 | 15 | 6 | 16 | 15 | 20 | 20 | 20 | 20 |
| α-hydroxy | Glycolic acid | 3 | 0.6 | — | — | — | — | — | — | — |
| acid | Lactic acid | — | — | 3 | 2 | 1 | 5 | — | — | — |
| | Malic acid | — | — | — | — | — | — | 1.5 | — | — |
| | Tartaric acid | — | — | — | — | — | — | — | 1.5 | — |
| | Mandelic | — | — | — | — | — | — | — | — | 2.5 |
| | acid | | | | | | | | | |
| | Citric acid | — | — | — | — | — | — | — | — | — |
| | Acetic acid | — | — | — | — | — | — | — | — | — |
| | Hydrochloric | — | — | — | — | — | — | — | — | — |
| | acid | | | | | | | | | |
| Water | | 69.5 | 51.4 | 74 | 45 | 49 | 60 | 53.5 | 53.5 | 52.5 |
| Test items | Solubility | ∘ | ∘ | ∘ | ∘ | ∘ | ∘ | ∘ | ∘ | ∘ |
| | Stability | ∘ | ∘ | ∘ | ∘ | ∘ | ∘ | ∘ | ∘ | ∘ |

TABLE 2

| Composition | | Embodiment | | | Comparative Example | | | | | |
|---|---|---|---|---|---|---|---|---|---|---|
| (w/w %) | | 10 | 11 | 12 | 1 | 2 | 3 | 4 | 5 | 6 |
| Minoxidil | | 5 | 5 | 5 | 5 | 5 | 5 | 5 | 5 | 5 |
| Cosolvent | Ethanol | 20 | 20 | — | 10 | 30 | 10 | 5 | 20 | 15 |
| | Isopropyl alcohol | — | — | 20 | — | — | — | — | — | — |
| | Glycerol | 20 | 20 | 20 | 5 | 15 | 5 | 10 | 10 | 15 |
| α-hydroxy | Glycolic acid | — | — | 1.5 | 1 | 0.5 | — | — | — | — |
| acid | Lactic acid | — | — | — | — | — | 2 | 2.5 | 0.5 | — |
| | Malic acid | — | — | — | — | — | — | — | — | — |
| | Tartaric acid | — | — | — | — | — | — | — | — | — |
| | Mandelic acid | — | — | — | — | — | — | — | — | — |
| | Citric acid | 2.5 | — | — | — | — | — | — | — | — |
| | Acetic acid | — | 2.5 | — | — | — | — | — | — | — |
| | Hydrochloric acid | — | — | — | — | — | — | — | — | 2.5 |
| Water | | 52.5 | 52.5 | 53.5 | 79 | 49.5 | 78 | 77.5 | 64.5 | 62.5 |
| Test items | Solubility | ∘ | ∘ | ∘ | x | x | x | x | x | x |
| | Stability | ∘ | ∘ | ∘ | x | x | x | x | x | x |

It may be seen from the results in Table 1 and Table 2 that compared to Comparative Example 1, Comparative Example 3 and Comparative Example 4 in which the cosolvent content is less than 18 weight percent concentration, Embodiment 1 to Embodiment 12 in which the content of the cosolvent is between 18 weight percent concentration and 50 weight percent concentration have better solubility and better stability.

It may be seen from the results in Table 1 and Table 2 that compared to Comparative Example 2 and Comparative Example 5 in which the α-hydroxy acid content is less than 0.6 weight percent concentration, Embodiment 1 to Embodiment 12 in which the content of the α-hydroxy acid is between 0.6 weight percent concentration and 5 weight percent concentration have better solubility and better stability.

It may be seen from the results in Table 1 and Table 2 that compared to Comparative Example 6 that does not use α-hydroxy acid (e.g., Comparative Example 6 that uses 0.001M hydrochloric acid), Embodiment 1 to Embodiment 12 that use α-hydroxy acid have better solubility and better stability.

It may be seen from the results in Table 1 and Table 2 that compared to Comparative Example 4 in which the weight ratio of alcohol to water is 1:15.5, Embodiment 1 to Embodiment 12 in which the weight ratio of alcohol to water is between 1:1.4 and 1:10 have better solubility and better stability.

### <Manufacture of Topical Composition in the Gel or Foam Form>

### Embodiment 13

First, lactic acid was dissolved in water to obtain an acid solution. Next, ethanol and glycerol were added to the acid solution to obtain a mixed solution. Then, minoxidil was added to the mixed solution and stirred until the minoxidil was dissolved. Next, cocamidopropyl betaine was added, and then water was added until reaching the final required weight to obtain the topical composition in foam form of Embodiment 13. Based on the total weight of the topical composition of Embodiment 13 (i.e., when the topical composition of Embodiment 13 is considered as 100 weight percent concentration), the contents of minoxidil, ethanol, glycerol, lactic acid, cocamidopropyl betaine, and water are 5%, 20%, 20%, 2.5%, 1%, and 51.5% respectively, as shown in Table 3.

### Embodiment 14

The topical composition of Embodiment 14 is prepared by similar steps as Embodiment 13, with the difference being the types and amounts of ingredients used in Embodiment 14, and hydroxyethyl cellulose was also added in Embodiment 14 to obtain a topical composition in the foam form. The types and amounts of ingredients used in Embodiment 14 are listed in Table 3.

### Embodiment 15

The topical composition of Embodiment 15 is prepared by similar steps as Embodiment 13, with the difference being the types and amounts of ingredients used in Embodiment 15, and that Embodiment 15 did not use cocamidopropyl betaine, but hydroxyethyl cellulose was added to Embodiment 15 to obtain a topical composition in the gel form. The types and amounts of ingredients used in Embodiment 15 are listed in Table 3.

TABLE 3

| Composition | | Embodiment | | |
|---|---|---|---|---|
| (w/w %) | | 13 | 14 | 15 |
| Minoxidil | | 5 | 5 | 5 |
| Cosolvent | Ethanol | 20 | 20 | 20 |
| | Glycerol | 20 | 20 | 20 |
| α-hydroxy | Glycolic acid | — | — | 1.5 |
| acid | Lactic acid | 2.5 | 2.5 | — |
| Thickening | Hydroxyethyl cellulose | — | 0.5 | 1.5 |
| agent | | | | |
| Foaming | Cocamidopropyl betaine | 1 | 1 | — |
| agent | | | | |
| Water | | 51.5 | 51 | 52 |

To sum up, in the topical composition and the manufacturing method thereof according to one embodiment of the invention, the solubility and stability of minoxidil in water may be increased through the use of α-hydroxy acid and alcohol-containing cosolvent. Since there is no need to use propylene glycol, skin irritation caused by the topical composition may also be reduced, and skin allergies may be prevented.

Although the invention has been described in detail with reference to the above embodiments, they are not intended to limit the invention. Those skilled in the art should understand that it is possible to make changes and modifications without departing from the spirit and scope of the invention. Therefore, the protection scope of the invention shall be defined by the following claims.

What is claimed is:

1. A topical composition, comprising:
minoxidil;
a cosolvent, comprising alcohol;
an α-hydroxy acid; and
water,
wherein a weight ratio of the alcohol to the water is 1:1.4 to 1:10.

2. The topical composition according to claim 1, wherein content of the minoxidil is 1 weight percent concentration to 5 weight percent concentration based on total weight of the topical composition.

3. The topical composition according to claim 1, wherein content of the cosolvent is 18 weight percent concentration to 50 weight percent concentration based on total weight of the topical composition.

4. The topical composition according to claim 1, wherein the cosolvent further comprises glycerol, and a weight ratio of the alcohol to the glycerol is 1:0.45 to 1:2.

5. The topical composition according to claim 1, wherein the alcohol comprises ethanol, isopropyl alcohol, or a combination thereof.

6. The topical composition according to claim 1, wherein content of the α-hydroxy acid is 0.6 weight percent concentration to 5 weight percent concentration based on total weight of the topical composition.

7. The topical composition according to claim 1, wherein a weight ratio of the α-hydroxy acid to the water is 1:10 to 1:100.

8. The topical composition according to claim 1, wherein the α-hydroxy acid comprises glycolic acid, lactic acid, acetic acid, citric acid, malic acid, mandelic acid, tartaric acid, or a combination thereof.

9. The topical composition according to claim 1, wherein the topical composition does not comprise propylene glycol.

10. The topical composition according to claim 1, wherein content of the minoxidil is 1 weight percent concentration to 5 weight percent concentration, content of the cosolvent is 18 weight percent concentration to 50 weight percent concentration, and content of the α-hydroxy acid is 0.6 weight percent concentration to 5 weight percent concentration.

11. The topical composition according to claim 1, further comprising:
a thickening agent, wherein content of the thickening agent is 0.1 weight percent concentration to 3.0 weight percent concentration based on total weight of the topical composition.

12. The topical composition according to claim 11, wherein the thickening agent comprises alginate, acacia, carrageenan, guar gum, karaya gum, locust bean gum, tragacanth, xanthan gum, carboxymethylcellulose or salts thereof, hydroxyethyl cellulose, methylcellulose, hydroxypropyl cellulose, hydroxypropyl methylcellulose, hydroxypropyl methylcellulose stearoxy ether, hyaluronic acid or salts thereof, or a combination thereof.

13. The topical composition according to claim 1, further comprising:
a foaming agent, wherein content of the foaming agent is 0.1 weight percent concentration to 3.0 weight percent concentration based on total weight of the topical composition.

14. The topical composition according to claim 13, wherein the foaming agent comprises sodium laureth sulfate, sodium dodecyl sulfate, ammonium lauryl sulfate, cocamide foaming agents, PEG-20 oleyl ether, lauryl glucoside, potassium cocoyl glycinate, or a combination thereof.

15. The topical composition according to claim 14, wherein the cocamide foaming agent comprises nonionic cocamide DEA, nonionic cocamidopropylamine oxide, zwitterionic cocamidopropyl betaine, zwitterionic cocamidopropyl hydroxysultaine, or a combination thereof.

16. The topical composition according to claim 1, wherein a pH value of the topical composition is 4 to 5.

17. A manufacturing method of a topical composition, comprising:
dissolving an α-hydroxy acid in water;
adding cosolvent to obtain a mixed solution; and
adding minoxidil to the mixed solution,
wherein the cosolvent comprises alcohol, and a weight ratio of the alcohol to the water is 1:1.4 to 1:10.

18. The manufacturing method of the topical composition according to claim 17, further comprising:
adding a thickening agent;
wherein content of the thickening agent is 0.1 weight percent concentration to 3.0 weight percent concentration based on total weight of the topical composition.

19. The manufacturing method of the topical composition according to claim 17, further comprising:
adding a foaming agent;
wherein content of the foaming agent is 0.1 weight percent concentration to 3.0 weight percent concentration based on total weight of the topical composition.

20. A use of the topical composition according to claim 1 for treating hair loss or promoting hair growth.
